(19) United States
(12) Patent Application Publication
(10) Pub. No.: US 2003/0114657 A1
(43) Pub. Date: Jun. 19, 2003

(54) Truncated reelin protein and DNA encoding the same

(76) Inventors: Katsuhiko Mikoshiba, Tokyo (JP); Hidenori Tabata, Chiba (JP); Kazunori Nakajima, Tokyo (JP)

Correspondence Address: FINNEGAN, HENDERSON, FARABOW, GARRETT & DUNNER, LLP, 1300 I STREET, NW, WASHINGTON, DC, 20005, US

(21) Appl. No.: 09/832,189

(22) Filed: Apr. 11, 2001

(30) Foreign Application Priority Data
Apr. 11, 2000 (JP) 2000-109954

(51) Int. Cl. C07H 21/04; C07K 14/705

(52) U.S. Cl. 536/23.5; 435/69.1; 435/320.1; 435/325; 530/350

(57) ABSTRACT

The present invention provides a truncated Reelin protein containing a F-spondin domain and a CR-50 recognition site but containing no repeat site of a Reelin protein, and a DNA encoding the truncated Reelin protein. The truncated Reelin protein and the DNA encoding this protein of the present invention can be utilized for treatment of diseases including agyria due to abnormal alignment of neurons.

### FIELD OF THE INVENTION

The present invention relates to a truncated isoform of a Reelin protein and a DNA encoding the truncated Reelin protein.

### BACKGROUND OF THE INVENTION

Neuroblasts born on the cerebral ventricle side migrate to their destinations following this developmental fate and achieve their final differentiation in the developmental stages of the central nerve system. The question is how neuroblasts recognize their destinations. Reeler mice have been long known as mutant mice which are very useful in studying this mechanism. In Reeler mutant mice, neuroblasts cannot migrate to their correct destinations, so that abnormal alignment of neuroblasts are found in various locations in the central nerve system including cerebrum, cerebellum, and hippocampus.

To identify a molecule deficient in a Reeler mutant mouse, we immunized a Reeler mutant mouse with a normal embryonic mouse brain, obtaining a monoclonal antibody CR-50 (Neuron 14, 899-912, 1995). Therefore, localized expression of CR-50 antigens in Cajal Retzius cells was found in cerebrum.

Then, a Reelin protein which is a causative gene of the Reeler mutant was cloned (Nature 374, 719-723, 1995, Genomics 26, 543-549, 1995, Nature Genetics 10, 77-82, 1995).

A Reelin protein is a huge extracellular protein, comprising a F-spondin domain having a certain degree of homology with F-spondin on its N-terminal side, and of eight Reelin repeats occupying the most of Reelin protein on its C-terminal side beyond the hinge region (Nature 374, 719-723, 1995). Each Reelin repeat contains an EGF-like motif so that Reelin protein is predicted to have features of extracellular matrix (ECM).

In addition, it was shown that a CR-50 antigen is a Reelin protein itself and can also inhibit the function of Reelin both in vitro and in vivo (J. Neurosci., 17, 23-31, 1997; Nature 385, 70-74, 1997; J. Neurosci., 17, 3599-3609, 1997; Proc. Natul. Acad. Sci. USA, 94, 8196-8201, 1997).

Furthermore, the recognition site of CR-50 is confirmed to be placed between a F-spondin domain and a Reelin repeat I (J. Neurosci., 17, 23-31, 1997).

Based on the above background, the N-terminal of Reelin is a site directly involved in Reelin function. Moreover, Reelin repeat is thought to localize Reelin proteins as ECM-like molecules in the vicinity of Reelin-producing cells, such as Cajal-Retzius cells, so as to give more localized signals.

### SUMMARY OF THE INVENTION

An object of the present invention is to confirm the presence of a Reelin (herein after referred as “Reelin”) protein in Amphibian whose brain is normally shown to have no laminated structure.

The inventors cloned Reelin homologous molecules of Xenopus and confirmed the presence of a truncated isoform produced by alternative splicing during the process. This isoform has a F-spondin domain and a CR-50 recognition site, but has no Reelin repeat (hereinafter referred to as “repeat site.”). Moreover, the inventors showed that this type of isoform is also present in a mouse. Since these isoforms have no repeat site, it is thought that they have no properties of an ECM-like molecule but play a role as humoral factors distributed distantly. The inventors completed this invention based on these understandings.

The present invention is summarized as follows.

(1) A truncated Reelin protein comprising an F-spondin domain and a CR-50 recognition site of a Reelin protein but containing no repeat site.

(2) The truncated Reelin protein of (1) which is derived from Xenopus or mice.

(3) The truncated Reelin protein of (2) which is either one of the following proteins (a) or (b):

(a) a protein consisting of an amino acid sequence represented by SEQ ID NO: 2,

(b) a protein consisting of an amino acid sequence differing from the amino acid sequence of SEQ ID NO: 2 by deletion, substitution, or addition of one or more amino acids, and having Reelin protein activity.

(4) The truncated Reelin protein of (2) which is either one of the following proteins (a) or (b):

(a) a protein consisting of an amino acid sequence represented by SEQ ID NO: 4,

(b) a protein consisting of an amino acid sequence differing from the amino acid sequence of SEQ ID NO: 4 by deletion, substitution, or addition of one or more amino acids, and having Reelin protein activity.

(5) A DNA encoding a truncated Reelin protein comprising an F-spondin domain and a CR-50 recognition site of a Reelin protein but contains no repeat site.

(6) The DNA of (5) which is derived from Xenopus or mice.

(7) The DNA of (6) encoding a truncated Reelin protein which is either one of the following proteins (a) or (b):

(a) a protein consisting of an amino acid sequence represented by SEQ ID NO: 2,

(b) a protein consisting of an amino acid sequence differing from the amino acid sequence of SEQ ID NO: 2 by deletion, substitution, or addition of one or more amino acids, and having Reelin protein activity.

(8) The DNA of (7), which is any one of the following DNAs (a) to (c):

(a) a DNA having a nucleotide sequence represented by SEQ ID NO: 1,

(b) a DNA hybridizing to a nucleic acid probe which comprises a sequence of 1456^{th} to 2273^{rd} nucleotide in the nucleotide sequence of SEQ ID NO: 1 under stringent conditions [for example, at 42° C. in the presence of 5×SSPE and 50% formamide (20×SSPE 3 M NaCl, 173 mM NaH₂PO₄, 25 mM EDTA)], and encoding a protein having Reelin protein activity.

(c) a DNA having a nucleotide sequence which is a degenerate sequence of that of the DNA (a) or (b).

(9) The DNA of (6) encoding either one of the following truncated Reelin proteins (a) or (b):

(a) A protein comprising an amino acid sequence represented by SEQ ID NO: 4,

(b) a protein comprising an amino acid sequence differing from the amino acid sequence of SEQ ID NO: 4 by deletion, substitution, or addition of one or more amino acids, and having Reelin protein activity.

(10) The DNA of (9) which is any one of the following DNAs (a) to (c):

(a) a DNA having a nucleotide sequence represented by SEQ ID NO: 3,

(b) A DNA hybridizing to a nucleic acid probe which comprises a sequence of 2053^{rd} to 2758^{th} nucleotides in the nucleotide sequence of SEQ ID NO: 3 under stringent conditions [for example, at 42° C. in the presence of 5×SSPE and 50% formamide (20×SSPE=3 M NaCl, 173 mM NaH₂PO₄, 25 mM EDTA)], and encoding a protein having Reelin protein activity, or

(c) A DNA having a nucleotide sequence which is a degenerate sequence of that of the DNA (a) or (b).

In this specification the term “Reelin protein” means a causative gene of Reeler mutant mice and an extracellular matrix protein comprising a signal peptide, F-spondin domain, CR-50 recognition site, and Reelin repeat.

The term “F-spondin domain” is a region that is shown to have homology with F-spondin on the N-terminal side of a Reelin protein.

The term “CR-50 recognition site” is a site that a CR-50 antibody recognizes in a mouse Reelin protein, and a region homologous to the CR-50 recognition site of mouse Reelin in a Reelin protein of other organisms. CR-50 antibodies can be prepared by the method described in Neuron 14, 899-912, 1995.

The term “repeat site” is a region on the C-terminal side of a Reelin protein that contains repeating units which consist of an amino acid sequence with an EGF-like motif at its center, and which are homologous to one other.

The term “Reelin protein activity” includes any biological and immunological action that a Reelin protein possesses. An example is the function of aligning neurons in their correct positions. This can be confirmed by the methods described in Nature 385, 70-74, 1997, J. Neurosci., 17, 3599-3609, 1997, and Proc. Natul. Acad. Sci. USA, 94, 8196-8201, 1997.

Reelin is an essential molecule in developing a normal laminated structure of cerebrum. Though no laminated structure is observed in Amphibian cerebrum, Xenopus Reelin was searched to confirm whether Reelin molecules are present in such an organism.

As a result of PCR using degenerate primers, PCR fragments of Xenopus Reelin (Xreelin) were obtained. Surprisingly, 3′-RACE analysis revealed the presence of a splicing mutant which contains most of the F-spondin domain and hinge region on the N-terminal side of Xreelin, but contains no repeat site. Expression of both an intact form of Xreelin and a truncated form of Xreelin started at the tail bud embryonic stage (st. 28) and continued until the end of the development into an adult. Northern blotting showed that a transcript of the intact Xreelin was about 12 kb, as in a mouse. On the other hand, a band of the truncated Xreelin was confirmed at a 3 to 4 kb-position. Moreover, transcripts of the intact Xreelin were detected in the primordiums of the olfactory bulb and optic tectum by in situ hybridization. At tadpole stage (st. 47), expression of the transcripts was also confirmed in the cerebellum. These results were almost identical to those for mice, but no expression was observed in the cerebrum. This is consistent with the fact that Xenopus brain has no laminated structure.

The truncated Reelin protein of this invention contains no Reelin repeat which accounts for approximately 85% of a Reelin protein. Reelin repeat is a region containing 8 repeats of a sequence having an EGF-like motif, suggesting that it binds with an extracellular matrix molecule having another EGF-like motif. Accordingly, Reelin repeat is a region required to localize Reelin proteins in extracellular matrix and a truncated isoform lacking this repeat site may diffuse farther in vivo.

For example, diseases including agyria, polymicrogyriam, and ectopic gray matter due to abnormal neuronal alignment can be treated by incorporating cDNA encoding truncated Reelin proteins of this invention into expression vectors, introducing the vectors into neuroblasts and nerve trunk cells and the like derived from the tissue of a patient, and transplanting those cells into the patient brain.

### SEQUENCE LISTING FREE TEXT

SEQ ID NOS: 5 to 17, 19, 20, and 22 to 28 show nucleotide sequences of primers.

SEQ ID NOS: 18 and 20 show nucleotide sequences of probes.

### BRIEF DESCRIPTION OF THE DRAWINGS

FIG. 1 shows nucleotide sequences of the intact form of a Xenopus Reelin protein (Xreelin) (A) and of the truncated isoform (B) and a putative amino acid sequence of a Xenopus Reelin protein. “*” indicates a stop codon. Amino acids within a region pointed with an arrow (▾) are contained in the intact form of Reelin protein but not in the truncated isoform. A polyadenylation signal located 15 nucleotides upstream of a polyadenylation site is underlined.

FIG. 2A shows multiple alignments of putative amino acids of Xenopus, mouse and human Reelin proteins. Putative signal peptides deduced based on Signalase software are underlined with broken lines. F-spondin domains are underlined with bold lines. Regions surrounded with frames contain amino acid residues common among all of the three types. Gaps inserted are shown with “−.” Amino acid residues identical to those of Xreelin are shown with ●.

FIG. 2B is a diagram showing a mouse Reelin protein, Xreelin and truncated Xreelin. Undefined regions are shown with a broken line. Regions enclosed with frames denote coding regions and lines denote noncoding regions.

FIG. 3A shows the results of Northern blot analysis on Xreelin. A bold arrow (←) indicates the intact form of a Reelin protein and an arrow ( [symbol] ) indicates the truncated form of a Reelin protein.

FIG. 3B shows the results of Western blot analysis on Xreelin. A bold arrow (←) shows a Xreelin protein of the same size as a mouse Reelin protein detected with a Reelin protein antibody 142. A thin arrow (←) indicates a Xreelin protein fragment slightly smaller than a mouse Reelin protein fragment processed with metallo proteinase. An arrow ( [symbol] ) indicates a protein with a size predicted to be the truncated form of a Reelin protein.

FIG. 4 shows the results of RT-PCR using RNA samples at various developmental stages. At the top of a panel are numbers of developmental stages of samples in each lane.

FIG. 5 shows the distribution of the intact form of Xreelin mRNA by in situ hybridization (FIG. 5A, B, C, D, E, F). Anti-sense probes to XdII (D) and Eomesodermin (F) were also used to define the region of striatum in telencephalon and mitral cell in olfactory bulb. The specimens employed were (A) st. 35/36 whole embryos, (B) st. 47 whole brain, (E, F) horizontal sections of st. 51 olfactory bulb, and coronal sections of st. 54 at the level of (C, D) telencephalon, (G) tactum, and (H) spinal cord.

a: antisense probe, cp: cerebellum primordium, ob: olfactory bulb, s: sense probe, tec: tectum, Scale bars: A, B, 500 μm; C, D, G, H, 100 μm; E. F, 200 μm.

FIG. 6 shows the comparison of expression pattern of the intact and truncated forms of Xreelin mRNA by in situ hybridization (A to C) and TaqMan PCR analysis (D). The intact (A) and truncated forms (B) of Xreelin mRNA in the horizontal section of st. 51 cerebellum were detected. The neighboring section was hybridized to sense probe of the intact form (C). Scale bars: 100 μm. The total RNA of seven parts of Xenopus brain (E) was subjected to TaqMan PCR analysis. Unfilled bars and filled bars indicate the copy number of intact and truncated forms, respectively.

### EXAMPLE

Now, a detailed description of the present invention will be given as follows. These examples are intended as an explanation, but do not limit the scope of the invention.

### Example 1

### Cloning of Xenopus Reelin (Xreelin)

Method

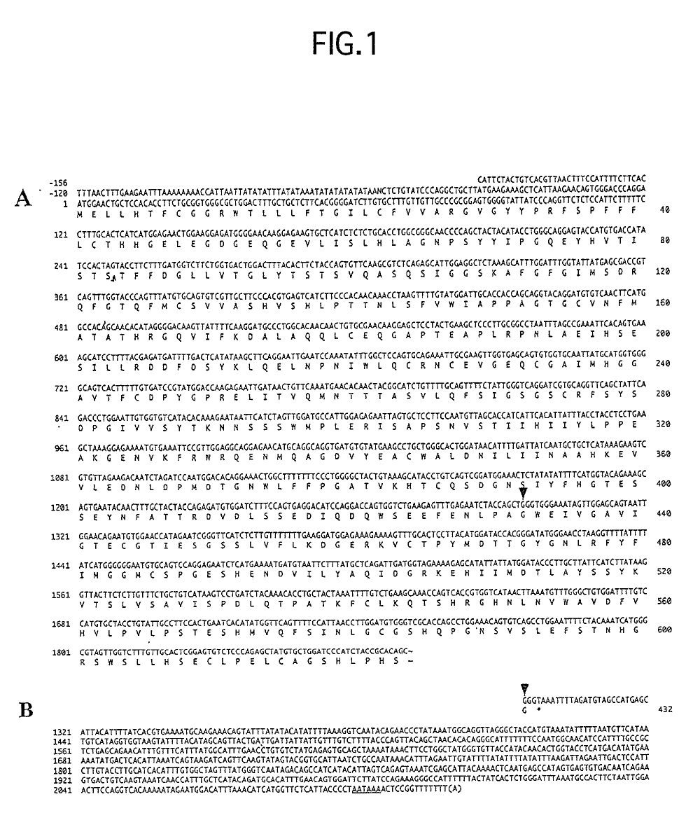

Random primed cDNA was synthesized from total RNA purified from stage (st.) 35 (Nieuwkoop, P. D. & Faber, J, 1967, in Normal Table of Xenopus laeis (Daudin), North-Holland Publishing Company, Amsterdam) Xenopus whole embryos using Super Script II (Gibco BRL), and subjected to PCR with degenerate primers. Using a set of degenerate primers [5′-A(A/G)TT(T/C)GGIAA(T/C)CA(A/G)TT(T/C)ATGTG-3′ (SEQ ID NO; 5) and 5′-TG(T/C)TCICCCAT(T/C)CA(A/G)TT-3′ (SEQ ID NO: 6)], the PCR fragment of Xreelin, which corresponds to 362 to 696 in the nucleotide sequence in FIG. 1 was obtained. To obtain the sequence further upstream from the PCR product, 5′ RACE (5′-rapid amplification of cDNA end) was performed. Poly (A)+RNA was purified from the total RNA isolated from st. 35 Xenopus whole embryo using a Fast Track 2.0 kit (Invitrogen). The later procedures were carried out using Gibco 5′ RACE system ver. 2 (GC rich protocol, Gibco BRL). First strand synthesis was primed with a gene specific primer [5′-ATGTCCTCACTGGAAAGATC-3′ (SEQ ID NO: 7)]. The purified product of second strand reaction was A-tailed, and PCR performed thereon using the AUAP primer (Gibco BRL) and gene specific primer [5′-CAGCAACACATAGGGGACAA-3′ (SEQ ID NO: 8)]. Moreover, 3′ RACE (3′-rapid amplification of cDNA end) was conducted using 3′ RACE system (Gibco BRL). First strand cDNA was generated by Super Script II with oligonucleotide [5′-GGCCACGCGTCGACTAGTACGAATTCATCTATAGC(T)₁₇-3′ (SEQ ID NO: 9)] from total RNA of st. 35 Xenopus whole embryos. PCR was performed with an adapter primer, the sequence of which is the complement of the oligonucleotide described above without (T)₁₇ and a gene specific primer [5′-CAGTGTCGTTGCTTCCCACGTGAGTCATCTTCCCA-3′ (SEQ ID NO: 10)]. This PCR product was further amplified with the adapter primer and a nested gene specific primer [5′-CGACAGGTACAGGATGTGTCAACTTCATGGCCACA-3′ (SEQ ID NO: 11)]. A single band was obtained from this step, and was cloned into pGEM-T Easy vector (Promega). By sequencing this clone, a truncated isoform generated by alternative splicing was identified. The intact form specific sequence was elucidated by screening of the cDNA library. The Poly (A)+RNA prepared by Fast Track 2.0 kit from st. 56 Xenopus tadpole was employed to first strand synthesis in addition to the use of oligo(dT)₁₂₋₁₈ and random hexamers. The synthesized cDNA was ligated to λ ZapII phage vector (Stratagene). The 1×10⁶ independent clones were screened by a ³²P labeled probe corresponding to 414 to 1253 nucleotides in the nucleotide sequence in FIG. 1. Thus 2 clones were isolated and sequenced. Sequencing of these clones revealed the nucleotide sequence of the intact form (1294 to 1869). The nucleotide sequences shown in FIG. 1 were confirmed by at least 3 rounds of separate PCR reactions.

Results

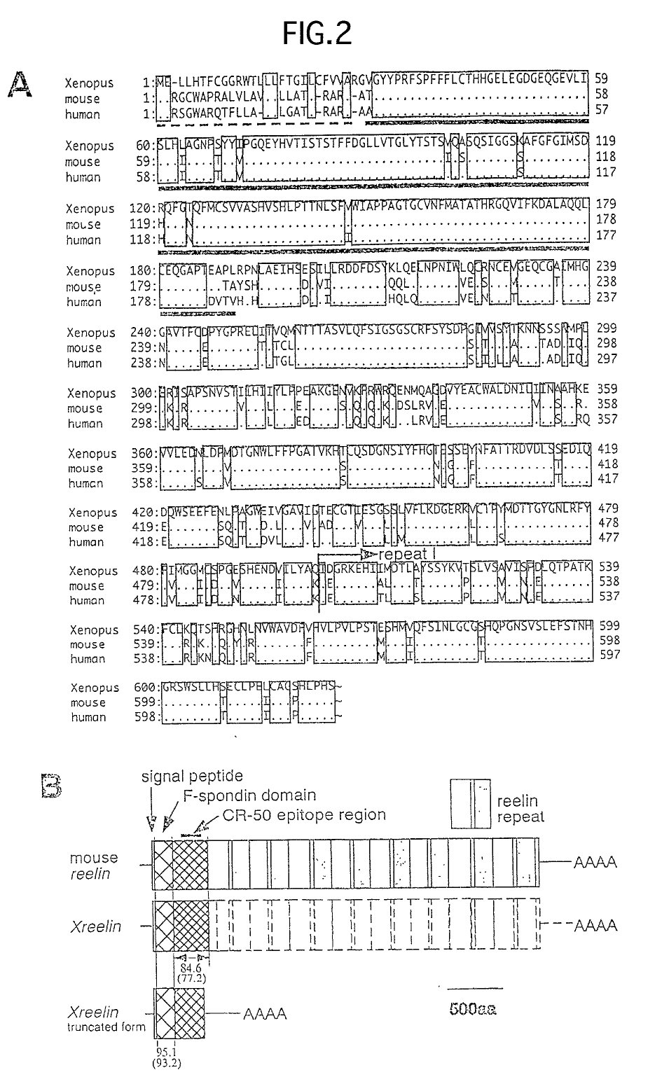

To obtain the Xenopus counterpart of Reelin, several sets of degenerate PCR primers were designed for various regions of the mouse Reelin. Among these primers, primer sets which enclose the boundary between the F-spondin domain and the CR-50 epitope region amplified a fragment with a sequence highly homologous to the mouse/human Reelin. Then 5′ RACE and screening of a cDNA library were performed so that the 5′-non-coding region of 156 base pairs (bp) and the coding region of 1873 bp were isolated (FIG. 1A). These sequences were confirmed by sequencing three independent PCR products. Comparison of the amino acid sequence of Reelin between the lower vertebrate and mouse/human clarified the region conserved in evolution (D' Arcangelo, G., Miao, G. G., Shu-Cheng, C., Soares, H. D., Morgan, J. I. & Curran, T., 1995, Nature 374, 719-723; DeSilva, U., D' Arcangelo G., Braden, V., Chen, J., Miao, G., Curran, T. & Green, E. D., 1997, Genome res. 7, 157-164). The deduced amino acid sequence of Xreelin is conserved as between mouse/human Reelin over the extent of the sequenced region, but especially in the F-spondin domain. The identity and similarity within the F-spondin domain are estimated 93.2% and 95.1%, respectively, whereas the identity and similarity of the hinge region, which contains the CR-50 epitope region and is between the F-spondin domain and Reelin repeats, 77.2% and 84.6%, respectively (FIGS. 2A and 2B). These results strongly suggest that the F-spondin domain is functionally important. This finding is particularly interesting as the inventors reported previously that CR-50, which recognizes just downstream of the F-spondin domain, blocks Reelin function both in vitro and in vivo (Ogawa, M., Miyata, T., Nakajima, K., Yagyu, K., Seike, M., Ikenaka, K., Yamamoto, H. & Mikoshiba, K., 19956, Neuron 14, 899-912; Del Rio, J. A., Heimrich, B., Borrell, V., Forster, E., Drakew, A., Alcantara S., Nakajima, K., Miyata, T., Ogawa, M., Mikoshiba, K., Derer, P., Frotscher, M. & Soriano, E., 1997, Nature 385, 70-74; Miyata, T., Nakajima, K., Mikoshiba, K. & Ogawa, M., 1997, J. Neurosci. 17, 3599-3609.; Nakajima, K., Mikoshiba, K., Miyata, T., Kudo, C. & Ogawa, M., 1997, Proc. Natl. Acad. Sci. USA 94, 8196-8201.

To obtain sequence information on downstream of the cloned region, 3′-RACE was performed. Thus, a truncated isoform of Xreelin containing only the F-spondin domain and the CR-50 epitope region was identified (FIGS. 1B and 2B). Interestingly and surprisingly, this novel isoform does not have any Reelin repeat. On this truncated isoform, the nucleotide sequence differs from that of the intact form from the second nucleotide of the TGG codon. That is, the TGG is changed to TAA. This difference coverts the amino acid, tryptophan 433, to a stop codon TAA. Thereafter, a non-coding region of 698 bp follows, and poly-adenylation signal (AATAAA) appears at 15 bp upstream of the poly-adenylation site. The nucleotides that differ between the intact form and the truncated form correspond to the end of exon #11 of mouse Reelin (Royaux, I., Lambert de Rouvroit, C., D' Arcangelo, G., Demirov, D. & Goffinet, A. M., 1997, Genomics 46, 240-250). Analysis of how this isoform is generated is not yet complete but the most likely explanation is the skipping of splicing and reading into intron #11. PCR amplification using a forward primer to both isoforms and a reverse primer specific only to the truncated form was done on either genomic DNA or random primed cDNA as a template. Both gave an amplified product of the same size, which is compatible with the skipping of splicing mechanism.

### Example 2

### Northern Blot Analysis

Method

Five grams of poly (A)+RNA prepared from st. 50, 56, and 60 Xenopus tadpole head was applied on each lane, subjected to electrophoresis, and then transferred to Zeta-Probe Blotting membrane (Bio-Rad). 32 P labeled RNA probes were synthesized by in vitro transcription with [α-32P] UTP and [α-32P] CTP using the sequence common to the intact and truncated form, and the 3′-noncoding region of the truncated form which corresponds to nucleotides 414-1253 and 1302-2099, respectively. Hybridization was carried out in 5×SSPE/50% formamide+5× Denhart's solution+0.5% SDS at 60° C. over night. The filter was washed twice in 2×SSC+0.1% SDS at 60° C. for 30 minutes, and then in 0.1×SSC+0.1% SDS at 60° C. for 30 minutes.

Results

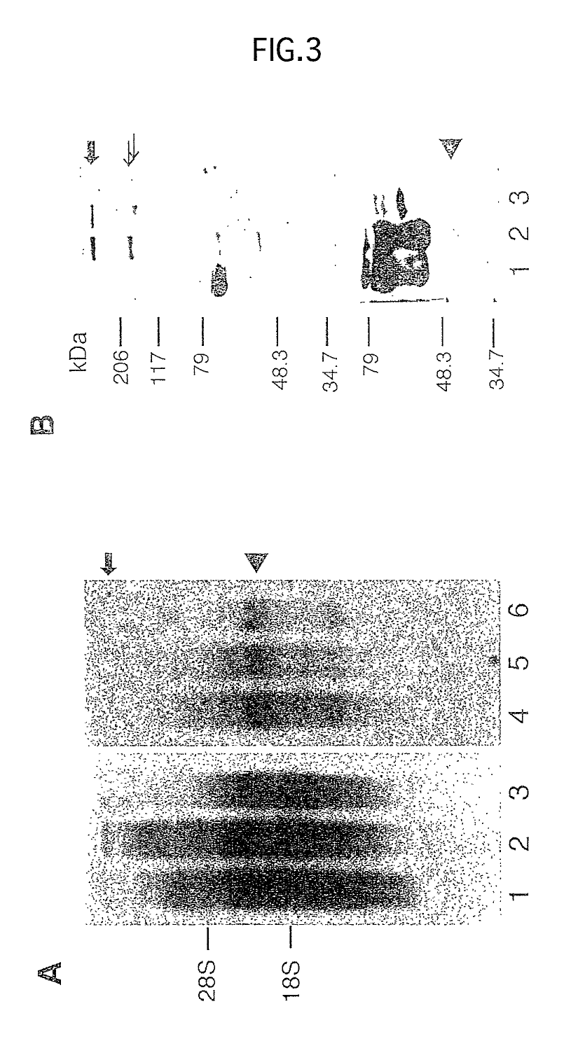

To confirm the presence of this novel isoform, Northern blot analysis was performed (FIG. 3). Using an RNA probe to a sequence common to both forms, a transcript of 12 to 13 kb was detected. This result indicates that there is indeed an intact form of Reelin in Xenopus as a huge molecule like that found in mice. Apart from this transcript, a transcript of 2.8 kb was also found. Moreover, when the 3′ non-coding region was used as a probe, only the shorter transcript of 2.8 kb was detected. The truncated form has a coding region of 1299 bases (b) and a 3′ untranslated region of 698 bases, and, so far, 156 bases of the 5′ untranslated region have been cloned and sequenced. Therefore, the mRNA of the truncated from is expected to consist of at least 2153 nucleotides. The size of the detected band on Northern blot analysis is larger than this size, but it is supposed that this discrepancy result from the unidentified region of the 5′ terminal untranslated region. Thus, it is concluded that mRNA of the truncated form certainly exists.

### Example 3

### Determination by RT-PCR of the Time of Xreelin mRNA Expression

Method

Total RNA was prepared from 2 cell stage and stage 7, 8, 10/11, 12.5, 13, 15, 19, 22, 28, 35/36, 42 and 50 of whole embryo and adult brain. Reverse transcription was performed from 1 g of total RNA, and 1/40 of the RT products was subjected to PCR using ³²P-dCTP. The PCR primers were designed in the common region between the intact and the truncated forms [5′-TCCCACAACAAACCTAAGTT-3′ (SEQ ID NO: 12) and 5′-ATGTCCTCACTGGAAAGATC-3′ (SEQ ID NO: 13)]. PCR for Histon H4 was also performed using the same templates as a control: [5′-CGGGATAACATTCAGGGTATCACT-3′ (SEQ ID NO: 14) and 5′-ATCCATGGCGGTAACTGTCTTCCT-3′ (SEQ ID NO: 15)]. The number of cycles was 24 and 19 for Xreelin and Histon H4, respectively.

Results

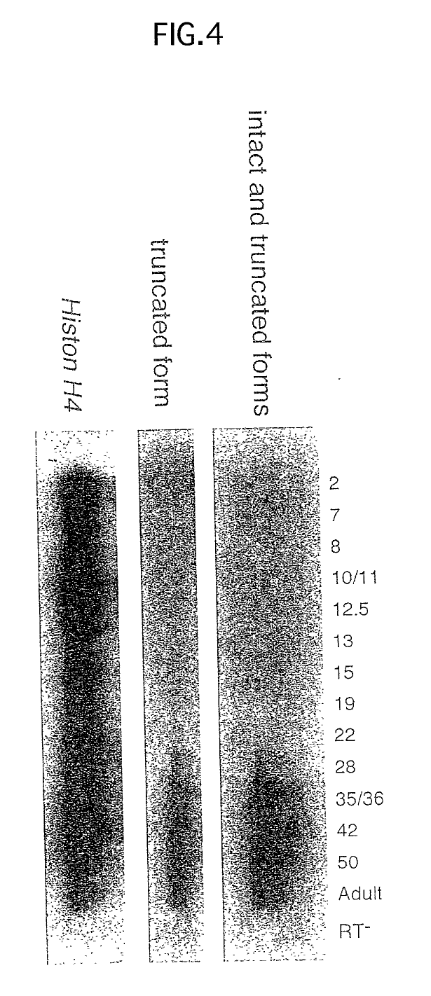

The time course of Xreelin expression at the transcription level was determined by RT-PCR (FIG. 4A). The primer set used in this assay was designed to amplify the common sequence between the intact form and the truncated form. To normalize each sample, PCR using a primer set for Histon H4 was performed. No Xreelin mRNA was detected during early development, and it first appeared in late neurula (st. 28). In tadpole stages, the signals became much stronger than those in neurula. Xreelin transcripts were also detected in the adult brain. In mouse development, Reelin mRNA becomes detectable after E8 by in situ hybridization and continues to be expressed in the adult brain (Ikeda, Y. & Terashima, T., 1997, Dev. Dyn. 210, 157-172; Schiffmann, S. N., Bernier, B. & Goffinet, A. M., 1997, European Journal of Neuroscience 9, 1055-1071.; Alcantara, S., Ruiz, M., D' Arcangelo, G., Ezan, F., de Lecea, L. & Curran, T., 1998, J. neurosci. 18, 7779-7799). Since the late neurula in Xenopus development is the switching point between neurulation and morphogenesis of the CNS, this stage corresponds to E8 of the mouse embryo in neural development. Therefore, the time course of Xreelin expression is similar to that of the mouse Reelin.

### Example 4

### Quantification of the Intact and the Truncated Form-mRNA Method

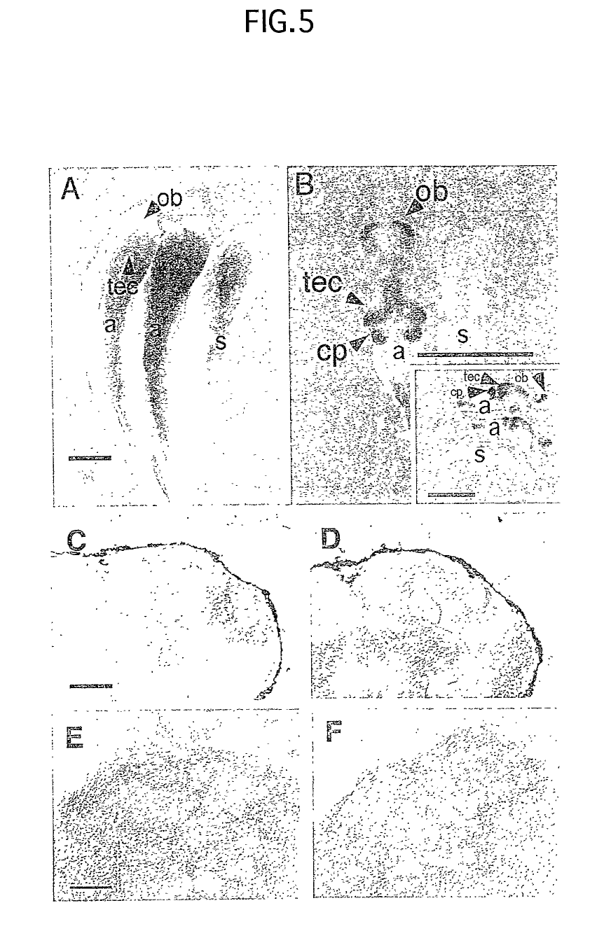

The amount of transcripts of the intact and truncated forms of Xreelin was evaluated by ABI PRISM 7700 (Perkin Elmer) using the gene-specific primers and oligonucleotide probes coupled with FAM (5-carboxyfluorescein) at the 5′ end and TAMRA (N, N, N′-tetramethyl-5-carboxyrhodamine) at the 3′ end (TaqMan probes). Total RNA was prepared from various stages (2-cell stage, st. 22, st. 28 and st. 43 whole embryos) and various parts of st. 50-54 brain (FIG. 5C). Reverse transcription (RT) was performed from 1 g of these RNA samples using Super Script II (Gibeo BRL) and random hexamer, and 1/40 of the products was subjected to PCR using TaqMan probes. The procedure for this PCR was according to the protocol of TaqMan PCR Reagent Kit (Perkin Elmer). At each point, the averages and standard deviations were obtained by three independent PCR from one RT-product. To detect the intact form, one set of primers [5′-GTCCTGATCTACAAACACCTGCTACT-3′ (SEQ ID NO: 16) and 5′-AGGTAGCACATGGACAAAATCC-3′ (SEQ ID NO: 17)] and a TaqMan probe [5′-(FAM)CTGAAGCAAACCAGTCACCGTGGTCA(TAMRA)-3′ (SEQ ID NO: 18)] were used. For the truncated form, the primer set [5′-TAGTGAGTGTGACAATCAGAAGTGA-3′ (SEQ ID NO: 19)] and [5′-GGCCCTTTCTGGATAAGAATC-3′ (SEQ ID NO: 20)], and a TaqMan-probe [5′-(FAM)TCAACCATTTGCTCATACAGATGCACA(TAMRA)-3′ (SEQ ID NO: 21)] were used. The copy numbers were estimated by a standard curve made from a dilution-series of plasmid DNA containing the intact form or the truncated form-specific sequence.

Results

Then the developmental profile of the truncated in comparison with the intact form was examined. To address this issue, the amount of intact form and truncated form RNA was determined at several time-points (FIG. 4B). The amount of RNA was quantified by the TaqMan PCR technique using probes labeled with fluorescent dye, FAM and TAMRA, on the 5′ and 3′ ends, and then the degradation of the probe was observed during PCR. Expression of the truncated from is first observed around st. 28 and become much stronger in the later stages. This expression pattern resembles that of the intact form, and the proportion of the amount of truncated form versus intact form is constantly 5 to 10% throughout development. These results indicate that the expression profile of the both forms during development is regulated in the same way, although the absolute amounts differ.

### Example 5

### Analysis of Xreelin mRNA Localization by In Situ Hybridization

Method

The SDS-based in situ hybridization protocol was used (Shain, D. H. & Zuber, M. X., 1996, J. Biochem. Biophys. Methods 31, 185-188). Whole embryos of st. 35/36 were fixed in MEMFA buffer (Harland, R. M. in Methods in Cell biology, 1991, Academic Press Inc., San Diego, pp685-694) for 90 min. The brains of st.47 were dissected out in MEMFA and fixed in fresh MEMFA for 90 min. The digoxygenin labeled-RNA probe was synthesized by in vitro transcription using plasmid DNA containing the nucleotide sequence corresponding to 414-1252 in FIG. 1A as a template. The alkaline phosphatase chromogenic reaction was carried out in Purple AP (Boehringer-Manheim) for several hours at room temperature. The brain of st. 51 was dissected out in 4% paraformaldehyde in 70% PBS, and fixed in fresh same fixative overnight. The specimens embedded in OCT compound (Tissue Tech) were sectioned in 20 μm thick, and employed to in situ hybridization on the same way as for the whole embryos/brains with the exception that the NBT/BCIP solution in alkaline phosphatase buffer was used in the step of chromogenic reaction. RNA probes to the intact and truncated forms were synthesized from the sequences corresponded to 1296 to 1825 in FIGS. 1A and 1302 to 2099 in FIG. 1B, respectively. RNA probes to XdII and eomesodermin were prepared from the plasmid containing the PCR fragment obtained by using each gene-specific primers [5′-CCTCCAAGTCTGCCTTTATG-3′ (SEQ ID NO: 22) and 5′-GCGGACAACAATATGCAAGG-3′ (SEQ ID NO: 23)] for XdII, and [5′-GCGGACAACAATATGCAAGG-3′ (SEQ ID NO: 24) and 5′-GGTTGTTGACAAACTGGTCC-3′ (SEQ ID NO: 25)] for eomesodermin from the cDNA prepared from st. 51 Xenopus tadpole.

Results

Reelin molecule is required for well-arranged lamination in the mouse brain development. Whereas the Reelin counterpart exists in Xenopus, its telencephalon shows no obvious laminated structure. Therefore, it is important whether or not Xreelin is expressed in the Xenopus dorsal pallium, which is a homologue of the mouse neocortex (Northcutt, G. R. & Kaas, J. H., 1995, Trends Neurosci 18, 373-379,; Fernandez, A. S., Pieau, C., Reperant, J., Boncinelli, E. & Wassf, M., 1998, Development 125, 2099-2111). To address this point, the distribution of Xreelin mRNA in telencephalon was examined more precisely. XdII is a Xenopus counterpart of distalless (Asano, M., Emori, Y., Saigo, K. & Shiokawa, K., 1992, J. Biol. Chem. 267, 5044-5047), and known to be expressed in the striatum (Asano, M., Emori, Y., Saigo, K. & Shiokawa, K., 1992, J. Biol. Chem. 267, 5044-5047). XdII mRNA is localized in the ventral side of telencephalon (FIG. 5D), and the Xreelin transcript is found more dorsally (lateral pallium) to the XdII-positive region (FIG. 5C). In the dorsal pallium, which is lying dorsally to the lateral pallium, Xreelin is expressed weakly in a few scattered cells near the surface of the telencephalic vesicle. These cells might correspond to the Cajal-Retzois cells of the mouse neocortex and have some function in the morphogenetic events other than the multi-layer alignment of neuroblasts.

In developing olfactory bulb of a mouse, the Reelin transcript is expressed in mitral cells. The Xreelin-expressing cells were identified in the olfactory bulb in Xenopus. Eomesodermin (Eomd) is known to be specifically expressed in mitral cells of the olfactory bulb (Ryan, K., Garett, N., Mitchell, A. & Gurdon, J. B., 1996, Cell 87, 989-1000; Ryan, K., Butler, K., Bellefroid, E. & Burdon, J. B., 1998, Mech. Dev. 75, 167-170). Using a probe to Eomd, it was clarified that the mitral cells are arranged in a “V” shaped pattern on horizontal sections of st. 51 olfactory bulb (FIG. 5F), and the Xreelin transcript is detected in the same pattern (FIG. 5E). In consequently, it is concluded that Xreelin is expressed in mitral cells in the developing olfactory bulb.

Whole mount in situ hybridization of brains revealed that Xreelin is expressed in the tectum (FIG. 5B). To elucidate the localization of Xreelin-expressing cells in detail, an Xreelin-specific probe was used to hybridize cross sections of st. 54 tectum. Xreelin was found beneath the stratum opticum, which is the most superficial layer of the tectum (FIG. 5B). The retinal axons coming through the stratum opticum turn into the underlying multiple cell-layers and terminate at a specific subset of lamina. So far, the distribution of mouse Reelin mRNA in the superior colliculus has not been reported in detail. However, homozygous Reeler mice show some abnormality in the retinotectal projection, whereas the cytoarchitecture itself is rather normal (Frost, D. O., Edwards, M. A., Sachs, G. M. & Caviness, V. J., 1986, Brain Res 393, 109-20). Taken together, these data suggest that Xreelin has some role in retinotectal projection during development.

In the spinal cord, Xreelin signals are found mainly in the medial to intermedio-lateral portions slightly ventral to the middle plane between the dorsal extremity and the ventral extremity. Weak signals are also detected in the dorsal horn (FIG. 5H). These expression patterns are similar to those in mice (Ikeda, Y. & Terashima, T., 1997, Dev. Dyn. 210, 157-172; Schiffmann, S. N., Bernier, B. & Goffinete, A. M., 1997, European Journal of Neuroscience 9, 1055-1071; Alcanara, S., Ruiz, M., D'Arcangelo, G., Ezan, F., De Lecea, L. & Curran, T., 1998, J. Neurosci. 18, 7779-7799).

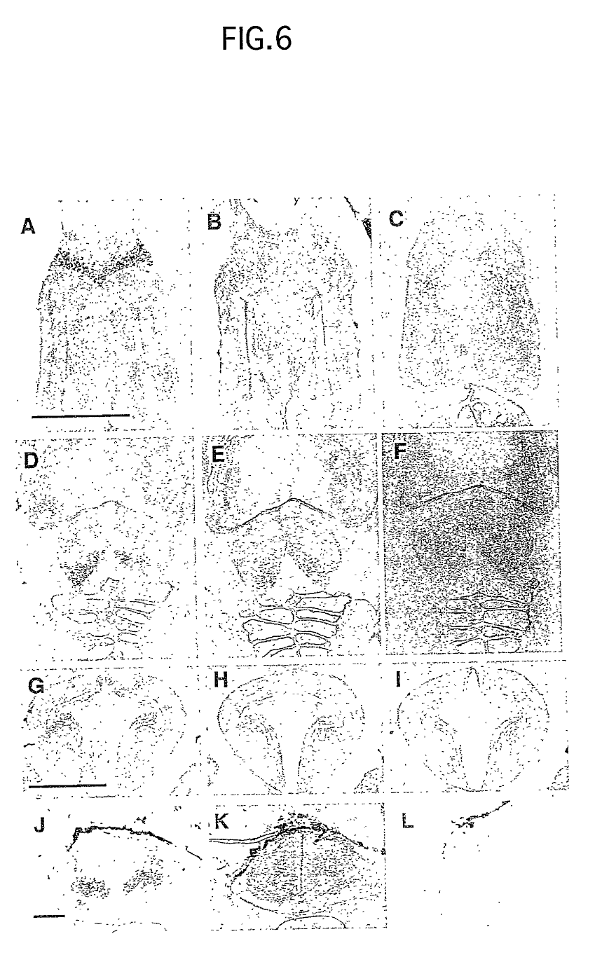

To investigate the localization of the truncated form of Xreelin, in situ hybridization was carried out using a truncated form-specific probe. Thus, faint signals were detected in the cerebellum (FIG. 6B) in a similar pattern to the intact form (FIG. 6A), but no in situ signal was detected in other regions. Next, TaqMan PCR analysis was performed using RNA from various brain regions of Xenopus (FIGS. 6C and 6D). A large amount of the intact form mRNA was found in the olfactory bulb, tectum and cerebellum, which was compatible with the expression pattern revealed by in situ hybridization. On the other hand, the truncated form of Xreelin was distributed in a similar manner to the intact form, and the ratio of the truncated form to the intact form was about 10% in each region. Considering the results obtained at various stages (FIG. 4B), it is likely that the expression profile of both forms is regulated by a common mechanism.

### Example 6

### Existence of Truncated Reelin Protein in Mice

Protocol

Mouse truncated Reelin protein was confirmed to exist by 3′-RACE. Total RNA was extracted from the spinal cord excised from a Reeler heterologous embryo on day 18 of the embryonal stage (the genotype had been specified by PCR). First strand synthesis using a primer [5′-GGCCACGCGTCGACTAGTACGAATTCATCTATAGC(T)₁₇-3′ (SEQ ID NO: 9)], and then PCR using an Adaptor primer AP2 [5′-CGCGTCGACTAGTACGAATT-3′ (SEQ ID NO: 26)] and a Reelin gene-specific primer RL-11 [5′-CTGATTGGATTCAGCTGGAG-3′ (SEQ ID NO: 27)] were performed. Further, the PCR product was subjected to nested PCR using AP2 and a Reelin gene-specific primer RL-12 [5′-ATTCAGCCCACAGAGAAGTC-3′ (SEQ ID NO: 28)].

Results

Three bands were confirmed by the PCR. The bands were separately incorporated into PGEM-T Easy vectors, and sequenced. Thus, one of them was an alternative splicing product which contains up to exon14 of mouse Reelin followed by an unknown sequence. The difference between the alternative splicing product and a full length Reelin protein is replacement of the final amino acid of exon14, serine by arginine immediately followed by a termination codon. Therefore, the alternative splicing product was determined to be the truncated form. At the end of the 3′-untranslated region of the mouse truncated Reelin, multiple poly-adenylation signals (AATAAA) were confirmed.

The truncated Reelin protein of this invention and DNA encoding the protein can be used for treatment of diseases including agyria due to abnormal neuronal alignment.

What is claimed is:

1. A truncated Reelin protein comprising an F-spondin domain and a CR-50 recognition site of a Reelin protein but containing no repeat site.

2. The truncated Reelin protein of claim 1 which is derived from Xenopus or mice.

3. The truncated Reelin protein of claim 2 which is either one of the following proteins (a) or (b):
(a) a protein consisting of an amino acid sequence represented by SEQ ID NO: 2, or
(b) a protein consisting of an amino acid sequence differing from the amino acid sequence of SEQ ID NO: 2 by deletion, substitution, or addition of one or more amino acids, and having Reelin protein activity.

4. The truncated Reelin protein of claim 2 which is either one of the following proteins (a) or (b):
(a) a protein consisting of an amino acid sequence represented by SEQ ID NO: 4, or
(b) a protein consisting of an amino acid sequence differing from the amino acid sequence of SEQ ID NO: 4 by deletion, substitution, or addition of one or more amino acids, and having Reelin protein activity.

5. A DNA encoding a truncated Reelin protein comprising an F-spondin domain and a CR-50 recognition site of a Reelin protein but containing no repeat site.

6. The DNA of claim 5 which is derived from Xenopus or mice.

7. The DNA of claim 6 encoding either one of the following truncated Reelin proteins (a) or (b):
(a) a protein consisting of an amino acid sequence represented by SEQ ID NO: 2, or
(b) a protein consisting of an amino acid sequence differing from the amino acid sequence of SEQ ID NO: 2 by deletion, substitution, or addition of one or more amino acids, and having Reelin protein activity.

8. The DNA of claim 7, which is any one of the following DNAs (a) to (c):
(a) a DNA having a nucleotide sequence represented by SEQ ID NO: 1,
(b) a DNA hybridizing to a nucleic acid probe which comprises a sequence of 1456th to 2273rd nucleotide in the nucleotide sequence of SEQ ID NO: 1 under stringent conditions, and encoding a protein with Reelin protein activity, or
(c) a DNA having a nucleotide sequence which is a degenerate sequence of that of the DNA (a) or (b).

9. The DNA of claim 6 encoding a truncated Reelin protein which is either one of the following proteins (a) or (b):
(a) a protein consisting of an amino acid sequence represented by SEQ ID NO: 4, or
(b) a protein consisting of an amino acid sequence differing from the amino acid sequence of SEQ ID NO: 4 by deletion, substitution, or addition of one or more amino acids, and having Reelin protein activity.

10. The DNA of claim 9 which is any one of the following DNAs (a) to (c):
(a) a DNA having a nucleotide sequence represented by SEQ ID NO: 3,
(b) a DNA hybridizing to a nucleic acid probe which comprises a sequence of 2053rd to 2758th nucleotide in the nucleotide sequence of SEQ ID NO: 3, and encoding a protein with Reelin protein activity, or
(c) a DNA having a nucleotide sequence which is a degenerate sequence of that of the DNA (a) or (b).


SEQUENCE LISTING (28 sequences)

SEQ ID NO: 1 - DNA, 2274 bp, Xenopus laevis
  misc_feature (100): a or g or t or c
cattctactgtcacgttaactttccattttcttcactttaactttgaagaatttaaaaaaaaccattaattatatatttatataaatatatatatataanctctgtatcccaggctgcttatgaagaaagctcattaagaacagtgggacccaggaatggaactgctccacaccttctgcggtgggcgctggactttgctgctcttcacggggatcttgtgctttgttgttgcccgcggagtggggtattatcccaggttctctccattctttttcctttgcactcatcatggagaactggaaggagatggggaacaaggagaagtgctcatctctctgcacctggcgggcaaccccagctactacatacctgggcaggagtaccatgtgaccatatccactagtaccttctttgatggtcttctggtgactggactttacacttctaccagtgttcaagcgtctcagagcattggaggctctaaagcatttggatttggtattatgagcgaccgtcagtttggtacccagtttatgtgcagtgtcgttgcttcccacgtgagtcatcttcccacaacaaacctaagttttgtatggattgcaccaccagcaggtacaggatgtgtcaacttcatggccacagcaacacataggggacaagttattttcaaggatgccctggcacaacaactgtgcgaacaaggagctcctactgaagctcccttgcggcctaatttagccgaaattcacagtgaaagcatccttttacgagatgattttgactcatataagcttcaggaattgaatccaaatatttggctccagtgcagaaattgcgaagttggtgagcagtgtggtgcaattatgcatggtggggcagtcactttttgtgatccgtatggaccaagagaattgataactgttcaaatgaacacaactacggcatctgttttgcagttttctattgggtcaggatcgtgcaggttcagctattcagaccctggaattgtggtgtcatacacaaagaataattcatctagttggatgccattggagagaattagtgctccttccaatgttagcaccatcattcacattatttacctacctcctgaagctaaaggagaaaatgtgaaattccgttggaggcaggagaacatgcaggcaggtgatgtgtatgaagcctgctgggcactggataacattttgattatcaatgctgctcataaagaagtcgtgttagaagacaatctagatccaatggacacaggaaactggctttttttccctggggctactgtaaagcatacctgtcagtcggatggaaactctatatattttcatggtacagaaagcagtgaatacaactttgctactaccagagatgtggatctttccagtgaggacatccaggaccagtggtctgaagagtttgagaatctaccagctgggtaaattttagatgtagccatgagcattacattttatcacgtgaaaatgcaagaaacagtatttatatacatattttaaaggtcaatacagaaccctataaatggcaggttagggctaccatgtaaatatttttaatgttcataatgtcataggtggtaagtattttacatagcagttactgattgattattattgtttgtcttttacccagttacagctaacacacagggcatttttttccaatggcaacatccattttgccgctctgagcagaacatttgtttcatttatggcatttgaacctgtgtctatgagagtgcagctaaaataaacttcctggctatgggtgttaccatacaacactggtacctcatgacatatgaaaaatatgactcacattaaatcagtaagatcagttcaagtatagtacggtgcattaatctgccaataaacatttagaattgtattttatattttatatttaagattagaattgactccattcttgtaccttgcatcacatttgtggctagtttatgggtcaatagacagccatcatacattagtcagagtaaatcgagcattacaaaactcaatgagccatagtgagtgtgacaatcagaagtgactgtcaagtaaatcaaccatttgctcatacagatgcacatttgaacagtggattcttatccagaaagggccattttttactatcactctgggatttaaatgccacttctaattggaacttccaggtcacaaaaatagaatggacatttaaacatcatggttctcattacccctaataaaactccggttttttta

SEQ ID NO: 2 - protein, 432 aa, Xenopus laevis
MELLHTFCGGRWTLLLFTGILCFVVARGVGYYPRFSPFFFLCTHHGELEGDGEQGEVLISLHLAGNPSYYIPGQEYHVTISTSTFFDGLLVTGLYTSTSVQASQSIGGSKAFGFGIMSDRQFGTQFMCSVVASHVSHLPTTNLSFVWIAPPAGTGCVNFMATATHRGQVIFKDALAQQLCEQGAPTEAPLRPNLAEIHSESILLRDDFDSYKLQELNPNIWLQCRNCEVGEQCGAIMHGGAVTFCDPYGPRELITVQMNTTTASVLQFSIGSGSCRFSYSDPGIVVSYTKNNSSSWMPLERISAPSNVSTIIHIIYLPPEAKGENVKFRWRQENMQAGDVYEACWALDNILIINAAHKEVVLEDNLDPMDTGNWLFFPGATVKHTCQSDGNSIYFHGTESSEYNFATTRDVDLSSEDIQDQWSEEFENLPAG

SEQ ID NO: 3 - DNA, 2745 bp, Mus musculus
  CDS (283)..(2052)
  sig_peptide (283)..(363)
  misc_feature (284)..(849): F-spondin domain
ggggcgtcgcgtgcacaccggcggcggcggcgctcggaggcggacgacgcgctctcggcgcccgcggccccggttccccccgcgctctcgctccggcggcccaaagtaacttcgggagcctcggtctcccgctaacttccccccgcgggctcggttgcccggacccgctcggctcgagcccgccgccggctcgccttccccgcacgcggctcctccgtgccggtgcctccgaaagtggatgagagagcgcgcggggcgcgcggcggcacggagcgcggcggcatggagcgcggctgctgggcgccgcgggctctcgtcctggccgtgctgctgctgctggcgacgctgagggcgcgcgcggccaccggctactacccgcgcttctcgcctttctttttcctgtgcacccaccacggggagctggaaggggatggggagcagggcgaggtgctcatttccctgcacattgcgggcaaccccacctactacgtaccgggacaggaataccatgttacaatttcaacaagcaccttctttgatggcttgctggtgacgggactctatacctcgacaagcatccagtcttctcagagcattggaggctccagcgcctttggattcgggatcatgtccgaccaccagtttggtaaccagtttatgtgcagtgtggtggcctctcatgtgagtcacctgcctacaaccaacctcagctttgtctggattgccccaccagctggcacaggctgtgtgaatttcatggctactgcaacacataggggccaggtgattttcaaagacgcactggcccagcagctgtgtgaacaaggagctcccacagaggccactgcttactcgcaccttgctgaaatacacagtgacagtgtgatcctacgagatgactttgactcctaccagcaactggaattgaaccccaacatatgggttgaatgcagcaactgtgagatgggagagcagtgtggcaccatcatgcatggcaatgctgtcaccttctgtgagccgtacggccctcgagagctgaccaccacatgcctgaacacaacaacagcatctgtcctccagttttccattgggtcaggatcatgtcgatttagttactctgaccccagcatcactgtgtcatacgccaagaacaataccgctgattggattcagctggagaaaattagagccccttccaatgtgagcacagtcatccacatcctgtacctccccgaggaagccaaaggggagagcgtgcagttccagtggaaacaggacagcctgcgagtgggtgaggtgtatgaggcctgctgggccctggataacatcctggtcatcaattcagcccacagagaagtcgttctggaggacaacctcgacccggtcgacacgggcaactggctcttcttccctggagcaacggtcaagcatagctgtcagtcagatgggaactccatttatttccatggaaatgaaggcagcgagttcaattttgccaccacccgggatgtagatctttctacagaggatattcaagagcagtggtcagaagaatttgagagccagcccacaggatgggatatcttgggagcagtagttggtgcagactgtggaaccgtagaatcaggactatcactggtgttcctcaaagatggagagaggaagctttgcaccccctacatggatacaactggttatggcaacctgaggttctacttcgttatgggaggaatctgtgaccctggagtctctcatgaaaacgatatcatcttatatgcaaagattgaaggaagaaaagaacacattgcactggacactcttacctattcttcctataaggttccgtctttggtttctgtggtcatcaaccctgaacttcagacacctgccaccaaattttgtctcaggcaaaagagccaccaagggtataatcggaatgtctgggctgtggacttcttccatgtgctgcccgttctcccttcaacaatgtctcacatgatccagttttctattaatttgggatgcggcacacaccagcctgggaacaggtgagaagcatgccgagtgtcctaacatggtaggaaataaacacatgcactggaccattgaagtaagtttgtcagtaggatttttggatgggattttaacaaaatatccattaagaaaatacagattcctactccctccctaaaagagttctttggttaataaatagaagggatgtgactgggtagatttttaggttagaatagtttcattcagggagcttgatacaagttatcagaggtgttcaccatgctgtgtggcagcatcccccgttctaacagattgctgggtgaagatgactgaagacaagattggcttctgttggctggtgaccccttataataggtatggaagtcaattagcacttcaagggctatgacttctctgctcctcttgcataagtgttgctcccatcctctgtaaagaactttgctgacctcacattcacaggatgaagtgacagtgtgagacatggtaattgcctagctatctatcaaattcaagagcacaaacccagtttactgtgtattgtccttcagacgtagcttttatggcagtaatccaatggcttgccctctgaaggctggtcaggcttcagtgagagatgacacatttagtaaaggtcttagagaaatcccacattcatcgactcattcaaggtatttagctagaaataaaaagaatcaaaaaaataaatta

SEQ ID NO: 4 - protein, 589 aa, Mus musculus
MERGCWAPRALVLAVLLLLATLRARAATGYYPRFSPFFFLCTHHGELEGDGEQGEVLISLHIAGNPTYYVPGQEYHVTISTSTFFDGLLVTGLYTSTSIQSSQSIGGSSAFGFGIMSDHQFGNQFMCSVVASHVSHLPTTNLSFVWIAPPAGTGCVNFMATATHRGQVIFKDALAQQLCEQGAPTEATAYSHLAEIHSDSVILRDDFDSYQQLELNPNIWVECSNCEMGEQCGTIMHGNAVTFCEPYGPRELTTTCLNTTTASVLQFSIGSGSCRFSYSDPSITVSYAKNNTADWIQLEKIRAPSNVSTVIHILYLPEEAKGESVQFQWKQDSLRVGEVYEACWALDNILVINSAHREVVLEDNLDPVDTGNWLFFPGATVKHSCQSDGNSIYFHGNEGSEFNFATTRDVDLSTEDIQEQWSEEFESQPTGWDILGAVVGADCGTVESGLSLVFLKDGERKLCTPYMDTTGYGNLRFYFVMGGICDPGVSHENDIILYAKIEGRKEHIALDTLTYSSYKVPSLVSVVINPELQTPATKFCLRQKSHQGYNRNVWAVDFFHVLPVLPSTMSHMIQFSINLGCGTHQPGNR

SEQ ID NO: 5 - DNA, 22 bp, Artificial Sequence
  Description of Artificial Sequencesynthetic DNA
arttyggnaaycarttyatgtg

SEQ ID NO: 6 - DNA, 16 bp, Artificial Sequence
  Description of Artificial Sequencesynthetic DNA
tgytccccatycartt

SEQ ID NO: 7 - DNA, 20 bp, Artificial Sequence
  Description of Artificial Sequencesynthetic DNA
atgtcctcactggaaagatc

SEQ ID NO: 8 - DNA, 20 bp, Artificial Sequence
  Description of Artificial Sequencesynthetic DNA
cagcaacacataggggacaa

SEQ ID NO: 9 - DNA, 51 bp, Artificial Sequence
  Description of Artificial Sequencesynthetic DNA
ggccacgcgtcgactagtacgaattcatctatagctttttttttttttttt

SEQ ID NO: 10 - DNA, 35 bp, Artificial Sequence
  Description of Artificial Sequencesynthetic DNA
cagtgtcgttgcttcccacgtgagtcatcttccca

SEQ ID NO: 11 - DNA, 35 bp, Artificial Sequence
  Description of Artificial Sequencesynthetic DNA
cgacaggtacaggatgtgtcaacttcatggccaca

SEQ ID NO: 12 - DNA, 20 bp, Artificial Sequence
  Description of Artificial Sequencesynthetic DNA
tcccacaacaaacctaagtt

SEQ ID NO: 13 - DNA, 20 bp, Artificial Sequence
  Description of Artificial Sequencesynthetic DNA
atgtcctcactggaaagatc

SEQ ID NO: 14 - DNA, 24 bp, Artificial Sequence
  Description of Artificial Sequencesynthetic DNA
cgggataacattcagggtatcact

SEQ ID NO: 15 - DNA, 24 bp, Artificial Sequence
  Description of Artificial Sequencesynthetic DNA
atccatggcggtaactgtcttcct

SEQ ID NO: 16 - DNA, 26 bp, Artificial Sequence
  Description of Artificial Sequencesynthetic DNA
gtcctgatctacaaacacctgctact

SEQ ID NO: 17 - DNA, 22 bp, Artificial Sequence
  Description of Artificial Sequencesynthetic DNA
aggtagcacatggacaaaatcc

SEQ ID NO: 18 - DNA, 26 bp, Artificial Sequence
  Description of Artificial Sequencesynthetic DNA
ctgaagcaaaccagtcaccgtggtca

SEQ ID NO: 19 - DNA, 25 bp, Artificial Sequence
  Description of Artificial Sequencesynthetic DNA
tagtgagtgtgacaatcagaagtga

SEQ ID NO: 20 - DNA, 21 bp, Artificial Sequence
  Description of Artificial Sequencesynthetic DNA
ggccctttctggataagaatc

SEQ ID NO: 21 - DNA, 27 bp, Artificial Sequence
  Description of Artificial Sequencesynthetic DNA
tcaaccatttgctcatacagatgcaca

SEQ ID NO: 22 - DNA, 20 bp, Artificial Sequence
  Description of Artificial Sequencesynthetic DNA
cctccaagtctgcctttatg

SEQ ID NO: 23 - DNA, 20 bp, Artificial Sequence
  Description of Artificial Sequencesynthetic DNA
gcggacaacaatatgcaagg

SEQ ID NO: 24 - DNA, 20 bp, Artificial Sequence
  Description of Artificial Sequencesynthetic DNA
gcggacaacaatatgcaagg

SEQ ID NO: 25 - DNA, 20 bp, Artificial Sequence
  Description of Artificial Sequencesynthetic DNA
ggttgttgacaaactggtcc

SEQ ID NO: 26 - DNA, 20 bp, Artificial Sequence
  Description of Artificial Sequencesynthetic DNA
cgcgtcgactagtacgaatt

SEQ ID NO: 27 - DNA, 20 bp, Artificial Sequence
  Description of Artificial Sequencesynthetic DNA
ctgattggattcagctggag

SEQ ID NO: 28 - DNA, 20 bp, Artificial Sequence
  Description of Artificial Sequencesynthetic DNA
attcagcccacagagaagtc